(19) United States
(12) Patent Application Publication
(10) Pub. No.: US 2003/0154507 A1
(43) Pub. Date: Aug. 14, 2003

(54) Synthetic herbicide resistance gene

(76) Inventors: Melvin J. Oliver, Lubbock, TX (US); John J. Burke, Lubbock, TX (US); Jeffrey P. Velten, Lubbock, TX (US)

Correspondence Address: USDA-ARS-OFFICE OF TECHNOLOGY TRANSFER, NATIONAL CTR FOR AGRICULTURAL UTILIZATION RESEARCH, 1815 N. UNIVERSITY STREET, PEORIA, IL, 61604, US

(21) Appl. No.: 10/279,452

(22) Filed: Oct. 24, 2002

Related U.S. Application Data
(60) Provisional application No. 60/335,463, filed on Oct. 24, 2001.
(60) Provisional application No. 60/375,529, filed on Apr. 25, 2002.

(51) Int. Cl. A01H 1/00; C12N 9/00; C12N 15/82; C07H 21/04; C12N 5/04; C12P 21/02

(52) U.S. Cl. 800/278; 435/69.1; 435/320.1; 435/419; 536/23.2; 435/183

(57) ABSTRACT

The invention provides a DNA molecule, a DNA construct, a transgenic plant and a transgenic plant part comprising a synthetic DNA sequence. The synthetic DNA sequence encodes an enzyme that degrades 2,4-dichlorophenoxyacetic acid (2,4-D) to dichlorophenol. The synthetic DNA sequence comprises a natural microbial sequence that encodes the enzyme in which at least a plurality of the codons of the natural microbial sequence have been replaced by codons more preferred by a plant. The invention also provides a method of controlling weeds in a field containing transgenic plants according to the invention by applying an auxin herbicide, such as 2,4-D, to the field. The invention further provides methods of selecting plants and plant cells that have been transformed with a DNA construct according to the invention using an auxin herbicide.

### CROSS-REFERENCE TO RELATED APPLICATIONS

This application hereby claims the benefit of U.S. provisional patent application serial No. 60/335,463, filed Oct. 24, 2001, and provisional patent application serial No. 60/375,529, filed Apr. 25, 2002, the contents of which are incorporated by reference herein.

### FIELD OF THE INVENTION

The present invention relates to a synthetic herbicide-resistance gene, its use to prepare herbicide-resistant transgenic plants and its use as a selection marker.

### BACKGROUND OF THE INVENTION

2,4-Dichlorophenoxyacetic acid (2,4-D) is a herbicide used to control broadleaf weeds. 2,4-D is degraded by Alcaligenes eutrophus and other microorganisms. The gene which encodes the first enzyme in the A. eutrophus 2,4-D degradation pathway is tfdA. This gene encodes a dioxygenase which catalyzes the conversion of 2,4-D to 2,4-dichlorophenol (DCP). DCP is much less toxic to plants than 2,4-D, and transgenic tobacco plants, cotton plants, and hardwood trees containing the tfda gene have been reported to have increased tolerance to 2,4-D. Streber et al., Bio/Technology, 7, 811-816 (1989); Lyon et al., Plant Molec. Biol., 13, 533-540 (1989); Bayley et al., Theor. Appl. Genet., 83, 645-649 (1992); Llewellyn and Last, in Herbicide-Resistant Crops, Chapter 10, pages 159-174 (Duke, ed.., CRC Press (1996)); Last and Llewellyn, Weed Science, 47, 401-404 (1999); U.S. Pat. Nos. 6,153,401, 6,100,446, and 5,608,147; and PCT applications WO 98/38294 and WO 95/18862. However, transgenic plants resistant to levels of 2,4-D that might be encountered in agricultural situations have not been obtained. See Last and Llewellyn, Weed Science, 47, 401-404 (1999). These authors suggest that codon optimization of the tfdA gene “might enhance tolerance levels.” Id. at 404. The tfdA gene has also been used as a selection marker to identify transformed plants and plant cells. U.S. Pat. No. 5,608,147; PCT application WO 95/18862.

### SUMMARY OF THE INVENTION

The invention provides a DNA molecule comprising a synthetic DNA sequence. The synthetic DNA sequence encodes an enzyme that degrades 2,4-dichlorophenoxyacetic acid to dichlorophenol. The synthetic DNA sequence comprises a natural microbial sequence that encodes the enzyme in which at least a plurality of the codons of the natural microbial sequence have been replaced by codons more preferred by a plant.

The invention also provides a DNA construct comprising the synthetic DNA sequence just described. In this construct, the synthetic DNA sequence is operatively linked to plant gene expression control sequences.

The invention further provides a transgenic plant or part of a plant. The transgenic plant or plant part comprises the synthetic DNA sequence operatively linked to plant gene expression control sequences.

The invention also provides a method of controlling weeds in a field containing transgenic plants according to the invention. The method comprises applying an amount of an auxin herbicide to the field effective to control the weeds in the field. The transgenic plants are tolerant to the auxin herbicide as a result of comprising and expressing the synthetic DNA sequence. Indeed, for the first time, transgenic plants have been produced which are tolerant to levels of auxin herbicides substantially greater than those normally used in agriculture for controlling weeds.

The invention further provides methods of selecting transformed plants and plant cells. The method of selecting transformed plant cells comprises providing a population of plant cells. At least some of the plant cells in the population are transformed with the DNA construct of the invention. Then, the resulting population of plant cells is grown in a culture medium containing an auxin herbicide at a concentration selected so that transformed plant cells proliferate and untransformed plant cells dol not proliferae.

The method of selecting transformed plants comprises providing a population of plants suspected of comprising a transgenic plant according to the invention. Then, an auxin herbicide is applied to the population of plants, the amount of herbicide being selected so that transformed plants will grow and growth of untransformed plants will be inhibited.

### BRIEF DESCRIPTION OF THE DRAWINGS

FIG. 1: Diagram of pProPClSV-SAD.

FIG. 2: Diagram of pPZP211-PNPT-311g7.

FIG. 3: Diagram of pPZP211-PNPT-512g7.

FIG. 4: Diagram of pPZP211-PNPT-311-SAD.

FIG. 5: Diagram of pPZP211-PNPT-512-SAD.

In these figures, SAD=2,4-D-degrading synthetic gene adapted for dicots; CDS=coding sequence; AMV-Leader=5′ untranslated leader sequence from the 35S transcript of alfalfa mosaic virus; PClSV-Promoter=peanut chlorotic streak virus promoter; T-Left=T-DNA left border from Agrobacterium tumefaciens nopaline Ti plasmid pTiT37; 35SPolyA=3′ polyadenylation (polyA) termination signal sequence from the cauliflower mosaic virus (CaMV) 35S transcript; NPTII=neomycin phosphotransferase II; g7PolyA=3′ polyA termination signal from gene 7 within the T-Left border of an A. tumefaciens octopine plasmid; MCS=multiple cloning site; T-Right=T-DNA right border from A. tumefaciens Ti plasmid pTiT37.

### DETAILED DESCRIPTION OF THE PRESENTLY-PREFERRED EMBODIMENTS OF THE INVENTION

The invention provides a synthetic DNA sequence. “Synthetic” is used herein to mean that the DNA sequence is not a naturally-occurring sequence.

The synthetic DNA sequence of the invention encodes an enzyme that degrades 2,4-dichlorophenoxyacetic acid (2,4-D) to dichlorophenol (DCP). The synthetic DNA sequence comprises a natural microbial sequence that encodes the enzyme, in which at least a plurality of the codons of the natural microbial sequence have been replaced by codons more preferred by a plant.

A “natural microbial sequence” is the coding sequence of a naturally-occurring microbial gene that encodes an enzyme that can degrade 2,4-D to DCP. Thus, the “natural microbial sequence” may be the coding sequence of a cDNA or genomic clone isolated from a microorganism, may be a chemically-synthesized DNA molecule having the same coding sequence as that of such a clone, or may be a combination of such sequences.

Multi-enzyme pathways for 2,4-D degradation have been demonstrated in several genera of bacteria. See, e.g., Lyon et al., Plant Molec. Biol., 13, 533-540 (1989), and references cited therein. Strains of Alcaligenes eutrophus have been the most extensively studied of these bacteria. The first enzyme in the A. eutrophus degradation pathway converts 2,4-D to DCP. This enzyme, which is often referred to as a monooxygenase, but which is now known to be a dioxygenase (see Fukumori et al., J. Bacteriol., 175, 2083-2086 (1993)), is encoded by the tfdA gene. Thus, the natural microbial sequence may be the coding sequence of a cDNA or genomic clone encoding a tfdA dioxygenase. Such clones and their isolation are described in Bayley et al., Theor. Appl. Genet., 83, 645-649 (1992), Lyon et al., Plant Molec. Biol., 13, 533-540 (1989), Streber et al., J. Bacteriology, 169, 2950-2955 (1987), Perkins and Lurquin, J. Bacteriology, 170, 5669-5672 (1988), and U.S. Pat. Nos. 6,100,446 and 6,153,401. See also Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989).

It is known that many bacteria are capable of degrading 2,4-D, including strains of Acinetobacter, Achromobacter, Alcaligenes, Arthrobacter, Corynebacterium, Flavobacterium, Pseudomona and strains of Actinomycetes (e.g., Nocardia spp. and Streptomyces viridochromogenes) (see, e.g., Llewellyn and Last, in Herbicide-Resistant Crops, Chapter 10 (Stephen O. Duke ed., CRC Press Inc. (1996)), Bayley et al., Theor. Appl. Genet., 83, 645-649 (1992), Lyon et al., Plant Molec. Biol., 13, 533-540 (1989), and Streber, et al., J. Bacteriology, 169, 2950-2955 (1987), Loos, in Degradation Of Herbicides, pages 1-49 (Kearney and Kaufman, eds., Marcel Dekker, Inc., New York 1969), and references cited in these references), and additional strains of bacteria that degrade 2,4-D can be isolated by methods well known in the art (e.g., by isolation from soils where 2,4-D is used by the enrichment culture technique) (see, e.g., Loos, in Degradation Of Herbicides, pages 1-49 (Kearney and Kaufman, eds., Marcel Dekker, Inc., New,York 1969)). Additional cDNA and genomic clones encoding an enzyme which converts 2,4-D to DCP can be obtained from these other bacteria in a similar manner as for the tfdA clones. See, e.g., Bayley et al., Theor. Appl. Genet., 83, 645-649 (1992); Lyon et al., Plant Molec. Biol., 13, 533-540 (1989); Streber et al., J. Bacteriology, 169, 2950-2955 (1987); Perkins and Lurquin, J. Bacteriology, 170, 5669-5672 (1988); U.S. Pat. Nos. 6,100,446 and 6,153,401. See also Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). In addition, or alternatively, isolated clones, portions of them, or sequences from them, can be used as probes to identify and isolate additional clones. See, e.g., Perkins and Lurquin, J. Bacteriology, 170, 5669-5672 (1988); Bayley et al., Theor. Appl. Genet., 83, 645-649 (1992); U.S. Pat. Nos. 6,100,446 and 6,153,401. See also Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). The natural microbial sequence may be the coding sequence of one of these cDNA or genomic clones.

It is also known that yeasts and fungi are capable of degrading 2,4-D (see, e.g., Llewellyn and Last, in Herbicide-Resistant Crops, Chapter 10 (Stephen O. Duke ed., CRC Press Inc. (1996)); Han and New, Soil Biol. Biochem., 26, 1689-1695 (1994); Donnelly et al., Applied And Environmental Microbiology, 59, 2642-2647 (1993); Loos, in Degradation Of Herbicides, pages 1-49 (Kearney and Kaufman, eds., Marcel Dekker, Inc., New York 1969), and references cited in these references), and additional strains of yeast and fungi that degrade 2,4-D can be obtained by methods well known in the art (e.g., by isolation from soils where 2,4-D is used by the enrichment culture technique) (see, e.g., Loos, in Degradation Of Herbicides, pages 1-49 (Kearney and Kaufman, eds., Marcel Dekker, Inc., New York 1969); Han and New, Soil Biol. Biochem., 26, 1689-1695 (1994)). Additional cDNA and genomic clones encoding an enzyme which converts 2,4-D to DCP can be obtained from yeast and fungi by methods well known in the art (see references cited above in the discussion of obtaining clones from bacteria), and the natural microbial sequence may be the coding sequence of one of these cDNA or genomic clones.

In addition, as noted above, the natural microbial sequence may be fully or partially chemically synthesized. To do so, a cDNA or genomic clone, obtained as described in the previous paragraphs, is sequenced by methods well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). A synthetic DNA sequence comprising the coding sequence of the cDNA or genomic clone can be fully or partially chemically synthesized using methods well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). For instance, DNA sequences may be synthesized by phosphoamidite chemistry in an automated DNA synthesizer. Also, the sequence of the tfdA gene from A. eutrophus JMP134 is publically available (see Streber et al., J. Bacteriology, 169, 2950-2955 (1987), U.S. Pat. Nos. 6,100,446 and 6,153,401, and GenBank (accession number M16730)), and a synthetic DNA sequence comprising the coding sequence of the A. eutrophus tfdA gene can also be fully or partially chemically synthesized.

The preferred natural microbial sequence is a natural bacterial sequence. A “natural bacterial sequence” is the coding sequence of a naturally-occurring bacterial gene that encodes an enzyme that can degrade 2,4-D to DCP. Thus, the “natural bacterial sequence” may be the coding sequence of a cDNA or genomic clone isolated from a bacterium, may be a chemically-synthesized DNA molecule having the same coding sequence as that of such a clone, or may be a combination of such sequences. Most preferably the natural bacterial sequence is the coding sequence of a cDNA or genomic clone isolated from a strain of A. eutrophus, a chemically-synthesized DNA molecule having the same coding sequence as that of such a clone, or a combination of such sequences.

As noted above, at least a plurality of the codons of the natural microbial sequence will be replaced by codons more preferred by a plant (also referred to herein as “plant-preferred codons”). A “codon more preferred by a plant” or a “plant-preferred codon” is a codon which is used more frequently by a plant to encode a particular amino acid than is the microbial codon encoding that amino acid. Preferably, the plant-preferred codon is the codon used most frequently by the plant to encode the amino acid. The plant codon usage may be that of plants in general, a class of plants (e.g., dicotyledonous plants), a specific type of plant (e.g., cotton or soybeans), etc. The codon usage or preferences of a plant or plants can be deduced by methods known in the art. See, e.g., Maximizing Gene Expression, pages 225-85 (Reznikoff & Gold, eds., 1986), Perlak et al., Proc. Natl. Acad. Sci. USA, 88, 3324-3328 (1991), PCT WO 97/31115, PCT WO 97/11086, EP 646643, EP 553494, and U.S. Pat. Nos. 5,689,052, 5,567,862, 5,567,600, 5,552,299 and 5,017,692. For instance, the codons used by the plant or plants to encode all of the different amino acids in a selection of proteins expressed by the plant or plants, preferably those proteins which are highly expressed, are tabulated. This can be done manually or using software designed for this purpose (see PCT application WO 97/11086).

The use of codons more preferred by the plant in which the synthetic DNA sequence will be expressed will improve expression as compared to use of the natural microbial sequence. The published reports indicate that codon usage affects gene expression in plants at the level of mRNA stability and translational efficiency. See, e.g., Perlak et al., Proc. Natl. Acad. Sci. USA, 88, 3324-3328 (1991); Adang et al., Plant Molec. Biol., 21:1131-1145 (1993); Sutton et al., Transgenic Res., 1:228-236 (1992). Not all of the codons of the natural microbial sequence need to be changed to plant-preferred codons in order to obtain improved expression. However, preferably at least the codons least preferred by the plant are changed to plant-preferred codons. “Codons least preferred by the plant” are those codons in the natural microbial sequence that are used least by the plant or plants in question to encode a particular amino acid. Preferably greater than about 50%, most preferably at least about 80%, of the microbial codons are changed to plant-preferred codons.

Plant-preferred codons can be introduced into the natural microbial sequence by methods well known in the art. For instance, site-directed mutagenesis can be used. See Perlak et al., Proc. Natl. Acad. Sci. USA, 88, 3324-3328 (1991). See also Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989). However, the plant-preferred codons are preferably introduced into the natural microbial sequence by chemically synthesizing the entire DNA sequence encoding the 2,4-D degrading enzyme. In particular, chemical synthesis is highly preferred when a large number of microbial codons are replaced by plant-preferred codons. In addition, chemical synthesis has a number of advantages. For instance, using chemical synthesis allows other changes to the sequence of the DNA molecule or its encoded protein to be made to, e.g., optimize expression (e.g., eliminate mRNA secondary structures that interfere with transcription or translation, eliminate undesired potential polyadenylation sequences, and alter the A+T and G+C content), add unique restriction sites at convenient points, delete protease cleavage sites, etc.

The synthetic DNA sequence having plant-preferred codons substituted for at least a plurality of microbial codons will encode the same amino acid sequence as the natural microbial sequence if these substitutions are the only differences in the sequence of the synthetic DNA sequence as compared to the natural microbial sequence. However, the synthetic DNA sequence may comprise additional changes as compared to the natural microbial sequence. For instance, the synthetic DNA sequence may encode an enzyme which degrades 2,4-D to DCP, but which has an altered amino acid sequence as compared to the enzyme encoded by the (unmutated) natural microbial sequence as a result of one or more substitutions, additions or deletions in the natural microbial sequence. Methods of making such substitutions, additions and deletions are well known in the art and are described above.

Assays for determining whether 2,4-D has been degraded to DCP are well known in the art. See, e.g., Streber et al., J. Bacteriol., 169, 2950-2955 (1987); Perkins et al., J. Bacteriol., 170, 5669-5672 (1988); Streber et al., Bio/Technology, 7, 811-816 (1989); Lyon et al., Plant Molec. Biol., 13, 533-540 (1989); Bayley et al., Theor. Appl. Genet., 83, 645-649 (1992); Fukumori et al., J. Bacteriol., 175, 2083 (1993); Lyon et al., Transgenic Res, 2, 162-169 (1993); Llewellyn and Last, in Herbicide-Resistant Crops, Chapter 10, pages 159-174 (Duke, ed.., CRC Press (1996)); Last and Llewellyn, Weed Science, 47, 401-404 (1999). Also, tolerance to 2,4-D and other auxin herbicides may be used to demonstrate this conversion. See below and references just cited.

The invention also provides DNA constructs comprising the synthetic DNA sequence operatively linked to plant gene expression control sequences. “DNA constructs” are defined herein to be constructed (non-naturally occurring) DNA molecules useful for introducing DNA into host cells, and the term includes chimeric genes, expression cassettes, and vectors.

As used herein “operatively linked” refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989).

“Expression control sequences” are DNA sequences involved in any way in the control of transcription or translation. Suitable expression control sequences and methods of making and using them are well known in the art.

The expression control sequences must include a promoter. The promoter may be any DNA sequence which shows transcriptional activity in the chosen plant cells, plant parts, or plants. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-61 (1987). Also, the location of the promoter relative to the transcription start may be optimized. See, e.g., Roberts, et al., Proc. Natl Acad. Sci. USA, 76, 760-4 (1979). Many suitable promoters for use in plants are well known in the art.

For instance, suitable constitutive promoters for use in plants include: the promoters from plant viruses, such as the peanut chlorotic streak caulimovirus (PClSV) promoter (U.S. Pat. No. 5,850,019), the 35S promoter from cauliflower mosaic virus (CaMV) (Odell et al., Nature 313:810-812 (1985)), promoters of Chlorella virus methyltransferase genes (U.S. Pat. No. 5,563,328), and the full-length transcript promoter from figwort mosaic virus (FMV) (U.S. Pat. No. 5,378,619); the promoters from such genes as rice actin (McElroy et al., Plant Cell 2:163-171 (1990)), ubiquitin (Christensen et al., Plant Mol. Biol. 12:619-632 (1989) and Christensen et al., Plant Mol. Biol. 18:675-689 (1992)), pEMU (Last et al., Theor. Appl. Genet. 81:581-588 (1991)), MAS (Velten et al., EMBO J. 3:2723-2730 (1984)), maize H3 histone (Lepetit et al., Mol. Gen. Genet. 231:276-285 (1992) and Atanassova et al., Plant Journal 2(3):291-300 (1992)), Brassica napus ALS3 (PCT application WO 97/41228); and promoters of various Agrobacterium genes (see U.S. Pat. Nos. 4,771,002, 5,102,796, 5,182,200, 5,428,147).

Suitable inducible promoters for use in plants include: the promoter from the ACE1 system which responds to copper (Mett et al. PNAS 90:4567-4571 (1993)); the promoter of the maize In2 gene which responds to benzenesulfonamide herbicide safeners (Hershey et al., Mol. Gen. Genetics 227:229-237 (1991) and Gatz et al., Mol. Gen. Genetics 243:32-38 (1994)), and the promoter of the Tet repressor from Tn10 (Gatz et al., Mol. Gen. Genet. 227:229-237 (1991). A particularly preferred inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al., Proc. Natl. Acad. Sci. USA 88:10421 (1991)) or the recent application of a chimeric transcription activator, XVE, for use in an estrogen receptor-based inducible plant expression system activated by estradiol (Zuo et al., The Plant Journal, 24:265-273 (2000)). Other inducible promoters for use in plants are described in EP 332104, PCT WO 93/21334 and PCT WO 97/06269.

Finally, promoters composed of portions of other promoters and partially or totally synthetic promoters can be used. See, e.g., Ni et al., Plant J., 7:661-676 (1995)and PCT WO 95/14098 describing such promoters for use in plants.

The promoter may include, or be modified to include, one or more enhancer elements. Preferably, the promoter will include a plurality of enhancer elements. Promoters containing enhancer elements provide for higher levels of transcription as compared to promoters that do not include them. Suitable enhancer elements for use in plants include the PClSV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 6, 143-156 (1997)). See also PCT WO 96/23898 and Enhancers And Eukaryotic Expression (Cold Spring Harbor Press, Cold Spring Harbor, N.Y., 1983).

For efficient expression, the coding sequences are preferably also operatively linked to a 3′ untranslated sequence. The 3′ untranslated sequence will include a transcription termination sequence and a polyadenylation sequence. The 3′ untranslated region can be obtained from the flanking regions of genes from Agrobacterium, plant viruses, plants or other eukaryotes. Suitable 3′ untranslated sequences for use in plants include those of the cauliflower mosaic virus 35S gene, the phaseolin seed storage protein gene, the pea ribulose biphosphate carboxylase small subunit E9 gene, the soybean 7S storage protein genes, the octopine synthase gene, and the nopaline synthase gene.

A 5′ untranslated sequence is also employed. The 5′ untranslated sequence is the portion of an mRNA which extends from the 5′ CAP site to the translation initiation codon. This region of the mRNA is necessary for translation initiation in plants and plays a role in the regulation of gene expression. Suitable 5′ untranslated regions for use in plants include those of alfalfa mosaic virus, cucumber mosaic virus coat protein gene, and tobacco mosaic virus.

As noted above, the DNA construct may be a vector. The vector may contain one or more replication systems which allow it to replicate in host cells. Self-replicating vectors include plasmids, cosmids and viral vectors. Alternatively, the vector may be an integrating vector which allows the integration into the host cell's chromosome of the synthetic DNA sequence encoding the 2,4-D-degrading enzyme. The vector desirably also has unique restriction sites for the insertion of DNA sequences. If a vector does not have unique restriction sites, it may be modified to introduce or eliminate restriction sites to make it more suitable for further manipulations.

The DNA constructs of the invention can be used to transform any type of plant cells (see below). A genetic marker must be used for selecting transformed plant cells (“a selection marker”). Selection markers typically allow transformed cells to be recovered by negative selection (i.e., inhibiting growth of cells that do not contain the selection marker) or by screening for a product encoded by the selection marker.

The most commonly used selectable marker gene for plant transformation is the neomycin phosphotransferase II (nptII) gene, isolated from Tn5, which, when placed under the control of plant expression control signals, confers resistance to kanamycin. Fraley et al., Proc. Natl. Acad. Sci. USA, 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, aminoglycoside-3′-adenyl transferase, and the bleomycin resistance determinant. Hayford et al., Plant Physiol. 86:1216 (1988), Jones et al., Mol. Gen. Genet. 210:86 (1987), Svab et al., Plant Mol. Biol. 14:197 (1990), Hille et al., Plant Mol. Biol. 7:171 (1986). Other selectable marker genes confer resistance to herbicides such as glyphosate, glufosinate or bromoxynil. Comai et al., Nature 317:741-744 (1985), Stalker et al., Science 242:419-423 (1988), Hinchee et al., Bio/Technology 6:915-922 (1988), Stalker et al., J. Biol. Chem. 263:6310-6314 (1988), and Gordon-Kamm et al., Plant Cell 2:603-618 (1990).

Other selectable marker genes for plant transformation are not of bacterial origin. These genes include, for example, mouse dihydrofolate reductase, plant 5-enolpyruvylshikimate-3-phosphate synthase, and plant acetolactate synthase. Eichholtz et al., Somatic Cell Mol. Genet. 13:67 (1987), Shah et al., Science 233:478 (1986), Charest et al., Plant Cell Rep. 8:643 (1990), EP 154,204.

Commonly used genes for screening presumptively transformed cells include β-glucuronidase (GUS), β-galactosidase, luciferase, and chloramphenicol acetyltransferase. Jefferson, R. A., Plant Mol. Biol. Rep. 5:387 (1987)., Teeri et al., EMBO J. 8:343 (1989), Koncz et al., Proc. Natl. Acad. Sci. USA 84:131 (1987), De Block et al., EMBO J. 3:1681 (1984), green fluorescent protein (GFP) (Chalfie et al., Science 263:802 (1994), Haseloff et al., TIG 11:328-329 (1995) and PCT application WO 97/41228). Another approach to the identification of relatively rare transformation events has been use of a gene that encodes a dominant constitutive regulator of the Zea mays anthocyanin pigmentation pathway. Ludwig et al., Science 247:449 (1990).

According to another aspect of the present invention, tolerance to an auxin herbicide can be used as a selection marker for plants and plant cells. “Auxin herbicide” is used herein to refer to phenoxy auxins (phenoxy herbicides), which include 2,4-D, 4-chlorophenoxyacetic acid, 4,-chloro-2-methylphenoxyacetic acid, 2,4,5-trichlorophenoxyacetic acid, 2,4-dichlorophenoxybutyric acid, 4-(2-methyl-4-chlorophenoxy)butryic acid, 2-(4-chlorophenoxy)propionic acid, 2-(2,4-dichlorophenoxy) propionic acid, 2-(2,4,5-trichlorophenoxy)propionic acid, and salts (including amine salts) and esters of these acids. Auxin herbicides are available commercially. See Crop Protection Reference (Chemical & Pharmaceutical Press, Inc., New York, N.Y., 11th ed., 1995). The preferred auxin herbicides are 2,4-D and its salts (including amine salts) and esters. “Tolerance” means that transformed plant cells are able to grow (survive, proliferate and regenerate into plants) when placed in culture medium containing a level of an auxin herbicide that prevents untransformed cells from doing so. “Tolerance” also means that transformed plants are able to grow after application of an amount of an auxin herbicide that inhibits the growth of untransformed plants.

Methods of selecting transformed plant cells are well known in the art. Briefly, at least some of the plant cells in a population of plant cells (e.g., an explant or an embryonic suspension culture) are transformed with a DNA construct comprising the synthetic DNA sequence of the invention. The resulting population of plant cells is placed in culture medium containing an auxin herbicide at a concentration selected so that transformed plant cells will grow, whereas untransformed plant cells will not. Suitable concentrations of an auxin herbicide can be determined empirically as is known in the art. At least in the case of 2,4-D, this amount may further need to be an amount which inhibits adventitious shoot formation from untransformed plant cells and allows adventitious shoot formation from transformed plant cells, since this is apparently the case with the natural-occurring bacterial tfdA gene. See U.S. Pat. No. 5,608,147 and PCT application WO 95/18862. In general, 2,4-D should be present in an amount ranging from about 0.001 mg/l to about 5 mg/l culture medium, preferably from about 0.01 mg/l to 0.2 mg/l culture medium.

Methods of selecting transformed plants are also known in the art. Briefly, an auxin herbicide is applied to a population of plants which may comprise one or more transgenic plants comprising a DNA construct of the invention providing for 2,4-D degradation. The amount of the auxin herbicide is selected so that transformed plants will grow, and the growth of untransformed plants will be inhibited. The level of inhibition must be sufficient so that transformed and untransformed plants can be readily distinguished (i.e., inhibition must be statistically significant). Such amounts can be determined empirically as is known in the art. See also Crop Protection Reference (Chemical & Pharmaceutical Press, Inc., New York, N.Y., 11^{th} ed., 1995).

Selection based on tolerance to an auxin herbicide can be used in the production of plants tolerant to 2,4-D and other auxin herbicides, in which case the use of another selection marker may not be necessary. Absence of a separate selection marker is advantageous since it minimizes the number of foreign genes expressed.

Selection based on tolerance to an auxin herbicide can also be used in the production of transgenic plants that express other genes of interest. Many such genes are known and include genes coding for proteins of commercial value and genes that confer improved agronomic traits on plants (see, e.g., PCT WO 97/41228, the complete disclosure of which is incorporated herein by reference).

The DNA constructs of the invention can be used to transform a variety of plant cells. The synthetic DNA sequence coding for the 2,4-D-degrading enzyme and the selection marker, if a separate selection marker is used, may be on the same or different DNA constructs. Preferably, they are arranged on a single DNA construct as a transcription unit so that all of the coding sequences are expressed together. Also, the gene(s) of interest and the synthetic DNA sequence coding for the 2,4-D-degrading enzyme, when tolerance to an auxin herbicide is being used as a selection marker, may be on the same or different DNA constructs. Such constructs are prepared in the same manner as described above.

Suitable host cells include plant cells of any kind (see below). Preferably, the plant cell is one that does not normally degrade auxin herbicides. However, the present invention can also be used to increase the level of degradation of auxin herbicides in plants that normally degrade such herbicides.

Thus, the “transgenic” plants, plant parts, and plant cells of the invention include plants, plant parts and plant cells that do not normally degrade auxin herbicides, but which have been transformed according to the invention so that they are able to degrade these herbicides, and progeny of such transformed plants, plant parts and plant cells. The “transgenic” plants, plant parts and plant cells of the invention also include plants, plant parts and plant cells that normally degrade auxin herbicides, but which have been transformed according to the invention so that they are able to degrade more of these herbicides or to degrade them more efficiently, and progeny of such transformed plants, plant parts and plant cells.

“Plant” should be understood as referring to a unicellular organism or a multicellular differentiated organism capable of photosynthesis, including algae, angiosperms (monocots and dicots), gymnosperms, bryophytes, ferns and fern allies. “Plant parts” are parts of multicellular differentiated plants and include seeds, pollen, embryos, flowers, fruits, shoots, leaves, roots, stems, explants, etc. “Plant cell” should be understood as referring to the structural and physiological unit of multicellular plants. Thus, the term “plant cell” refers to any cell that is a plant or is part of, or derived from, a plant. Some examples of cells encompassed by the present invention include differentiated cells that are part of a living plant, differentiated cells in culture, undifferentiated cells in culture, and the cells of undifferentiated tissue such as callus or tumors.

Methods of transforming plant cells are well known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized mechanism for introducing an expression vector into plants is based on the natural transformation systems of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10:1 (1991). Descriptions of Agrobacterium vector systems and methods for Agrobacterium-mediated gene transfer are provided by numerous references. See, for example, Horsch et al., Science 227:1229 (1985), Hoekema et al., Nature 303:179 (1983), de Framond et al., Bio/Technology 1:262 (1983), Jordan et al., Plant Cell Reports 7:281-284 (1988), Leple et al., Plant Cell Reports 11:137-141. (1992), Stomp et al., Plant Physiol. 92:1226-1232 (1990), Knauf et al., Plasmid 8:45-54 (1982)), Gruber et al., supra, Miki et al., supra, Moloney et al., Plant Cell Reports 8:238 (1989), PCT applications WO84/02913, WO84/02919 and WO84/02920, EP 116,718, and U.S. Pat. Nos. 4,940,838, 5,464,763, and 5,929,300.

A generally applicable method of plant transformation is microprojectile-mediated transformation wherein DNA is carried on the surface of microprojectiles. The expression vector is introduced into plant tissues with a biolistic device that accelerates the microprojectiles to speeds sufficient to penetrate plant cell walls and membranes. Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992), Klein et al., Nature 327:70-73 (1987).

Another method for physical delivery of DNA to plants is sonication of target cells. Zhang et al., Bio/Technology 9:.996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p. 53 (1990); D'Halluin et al., Plant Cell 4:1495-1505 (1992), Spencer et al., Plant Mol. Biol. 24:51-61 (1994), and Fromm et al., Proc. Natl. Acad. Sci. USA 82:5824 (1985). Other techniques include microinjection (Crossway, Mol. Gen. Genetics, 202:179-185 (1985)), polyethylene glycol transformation (Krens et al., Nature 296:72-74 (1982)), fusion of protoplasts with other entities, either minicells, cells, lysosomes, or other fusible lipid-surfaced bodies (Fraley et al., Proc. Natl. Acad. Sci. USA 79:1859-1863 (1982)), and techniques set forth in U.S. Pat. No. 5,231,019).

After selection, transformed plant cells are regenerated into transgenic plants. Plant regeneration techniques are well known in the art and include those set forth in the Handbook of Plant Cell Culture, Volumes 1-3, Evans et al., eds. Macmillan Publishing Co., New York, N.Y. (1983, 1984, 1984, respectively); Predieri and Malavasi, Plant Cell, Tissue, and Organ Culture 17:133-142 (1989); James, D. J., et al., J. Plant Physiol. 132:148-154 (1988); Fasolo, F., et al., Plant Cell, Tissue, and Organ Culture 16:75-87 (1989); Valobra and James, Plant Cell, Tissue, and Organ Culture 21:51-54 (1990); Srivastava, P. S., et al., Plant Science 42:209-214 (1985); Rowland and Ogden, Hort. Science 27:1127-1129 (1992); Park and Son, Plant Cell, Tissue, and Organ Culture 15:95-105 (1988); Noh and Minocha, Plant Cell Reports 5:464-467 (1986); Brand and Lineberger, Plant Science 57:173-179 (1988); Bozhkov, P. V. et al., Plant Cell Reports 11:386-389 (1992); Kvaalen and von Arnold, Plant Cell, Tissue, and Organ Culture 27:49-57 (1991); Tremblay and Tremblay, Plant Cell Tissue, and Organ Culture 27:95-103 (1991); Gupta and Pullman, U.S. Pat. No. 5,036,007; Michler and Bauer, Plant Science 77:111-118 (1991); Wetzstein, H. Y., et al., Plant Science 64:193-201 (1989); McGranahan, G. H., et al., Bio/Technology 6:800-804 (1988); Gingas, V. M., Hort. Science 26:1217-1218 (1991); Chalupa, V., Plant Cell Reports 9:398-401 (1990); Gingas and Lineberger, Plant Cell, Tissue, and Organ Culture 17:191-203 (1989); Bureno, M. A., et al., Phys. Plant. 85:30-34 (1992); and Roberts, D. R., et al., Can. J. Bot. 68:1086-1090 (1990).

Transgenic plants of any type may be produced according to the invention. Such plants include, for example, species from the genera Fragaria, Lotus, Medicago, Onobrychis, Trifolium, Trigonella, Vigna, Citrus, Linum, Ceranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Sencia, Salpiglossis, Cucumis, Browalia, Glycine, Lolium, Zea, Triticum, Sorghum, Malus, Apium, Datura and woody dicotyledonous forest tree species. In particular, broadleaf plants (including beans, soybeans, cotton, peas, potatoes, sunflowers, tomatoes, tobacco, fruit trees, ornamental plants and trees) that are currently known to be injured by auxin herbicides can be transformed so that they become tolerant to these herbicides. Other plants (such as corn, sorghum, small grains, sugarcane, asparagus, and grass) which are currently considered tolerant to auxin herbicides can be transformed to increase their tolerance to these herbicides.

In yet another embodiment, the invention provides a method of controlling weeds in a field where transgenic plants are growing. The method comprises applying an effective amount of an auxin herbicide to the field to control the weeds. Methods of applying auxin herbicides and the amounts of them effective to control various types of weeds are known. See Crop Protection Reference (Chemical & Pharmaceutical Press, Inc., New York, N.Y., 11th ed., 1995). For the first time, as a result of the present invention, transgenic plants have been produced which are tolerant to levels of auxin herbicides substantially greater than those normally used in agriculture for controlling weeds.

### EXAMPLES

### Example 1

Generation of Synthetic Plant-Optimized Sequence Encoding a 2,4-D Dioxygenase

The DNA sequence of a 2,4-D dioxygenase (also often referred to as a monooxygenase; see above) gene isolated from Alcaligenes eutrophus was obtained from the sequence database GenBank (accession number M16730). From this DNA sequence, the amino acid sequence of the protein coded for by the single open-reading frame (ORF) was determined [SEQ ID NO:1]. A codon usage table reflecting dicotyledonous ORFs was derived from a composite selection of random cDNA sequences from cotton, Arabidopsis and tobacco extracted from the GenBank database. A codon usage table reflecting monocotyledonous ORFs was derived from a random selection of cDNA sequences from maize, also extracted from the GenBank database. These are Tables 1 and 2 below. Using these plant-specific codon usage tables, the derived primary amino acid sequence of the bacterial 2,4-D dioxygenase was converted into DNA coding sequences that reflected the codon preferences of dicotyledonous and monocotyledonous plants [SEQ ID NOS:2 and 3, respectively].

The synthetic plant-optimized 2,4-D dioxygenase ORFs [SEQ ID NOS:2 and 3], both dicot and monocot, were then used to design 2,4-D dioxygenase genes capable of efficient expression in transgenic plants. These synthetic genes were designated as SAD (Synthetic gene Adapted for Dicots) and SAM (Synthetic gene Adapted for Monocots) for the dicot and monocot versions, respectively. In order to generate a translatable transcript once the gene had been constructed and inserted into a plant genome, a 5′ untranslated leader sequence representing the 5′ untranslated leader sequence from the 35S transcript of alfalfa mosaic virus (AMV; Gallie et al., Nucleic Acids Res., 15:8693-8711 (1987)) was incorporated into the design of the synthetic genes. In addition, a signature sequence, encoding Cys Ala Gly, was added to the 3′ end of the synthetic coding regions for each version of the synthetic gene. Finally, for ease of cloning, the designed sequences included a HindIII-specific overhang at the 5′ end and a SalI-specific overhang at the 3′ end. The complete designed sequences for the synthetic portions of the SAD and SAM genes are SEQ ID NOS:4 and 5.

To construct the designed synthetic portions of the SAD and SAM genes, each sequence was dissected into overlapping oligonucleotides, twelve oligonucleotides for each of the two strands resulting in a total of twenty-four oligonucleotides for each DNA sequence. A complete list of the oligonucleotides used to construct the synthetic portions of the SAD and SAM genes is given in Tables 3A, 3B, 4A, and 4B below. The oligonucleotides were synthesized using standard phosphoramidite chemistry by Integrated DNA Technologies, Inc., Coralville, Iowa. The synthetic DNA molecules were assembled using a procedure based upon the protocol described by Sutton et al. 1995 published on the World Wide Web (www.epicentre.com) using Ampliligase™ thermostable ligase (Epicentre Technologies Inc., Madison, Wis.). Oligonucleotides were first phosphorylated using T4 polynucleotide kinase (Invitrogen Life Technologies, Carlsbad, Calif.) as mixtures of upper and lower strand oligonucleotides for each synthetic DNA construct. Each mixture contained 10 pmoles of each oligonucleotide, 70 mM Tris/HCl pH 7.6, 10 mM MgCl₂, 5 mM dithiothreitol (DTT), 0.1 mM ATP, and 10 units of T4 polynucleotide kinase, for a total volume of 25 μl. Phosphorylation was achieved by incubation of the mixtures at 37° C. for 30 minutes, followed by a denaturing incubation at 70° C. for 10 minutes. To anneal and ligate the oligonucleotides, each reaction mixture was retreated at 70° C. for 10 minutes in a thermocycler and subsequently cooled to 65° C. over a 10-minute period. To each mixture, 65 μl of water, 10 μl of 10× Ampliligase buffer (Epicentre Technologies), and 2 μl of Ampliligase (5 units/μl) were added sequentially, and the temperature was reduced to 40° C. over a three hour period.

At this stage, in order to improve the efficiency of cloning, the complete synthetic DNA sequences for SAD and SAM were recovered from their respective annealing/ligation reactions by polymerase chain reaction (PCR) in an MJ Research Inc. (Waltham, Mass.) Model PTC-100 Thermocycler using Amplitaq Gold™ DNA polymerase under conditions supplied by the manufacturer, Perkin Elmer Life Sciences (Boston, Mass.). The PCR primers used for the recovery of each sequence were AGATCCTTTTTATTTTTAATTTTCTTTC [SEQ ID NO:6], a 28mer representing the 5′ end of the AMV leader sequence and used for both the SAD and SAM recovery PCR reactions, and CTCCAGCACACTAAACAACAGCGTC [SEQ ID NO:7], a 25mer specific for the 3′ end of the SAD sequence, and CTCCAGCACACTACACCACC [SEQ ID NO:8], a 20mer specific for the 3′ end of the SAM sequence. PCR fragments corresponding to the appropriate size of 918 bp were gel purified as described in Ausubel et al., Current Protocols In Molecular Biology (Green/Wiley Interscience, New York, 1989) and cloned between two XcmI restriction sites in pUCR19, a modified pUC19 vector designed for rapid cloning of PCR fragments using T overhangs generated by XcmI digestion (described in O'Mahony and Oliver, Plant Molecular Biology, 39:809-821 (1999)) to generate the plasmids pUCRsynSAD and pUCRsynSAM. Once cloned into these vectors, the inserts were sequenced to verify the sequence integrity of the designed synthetic portions of the SAD and SAM genes. DNA sequencing was performed by use of a dRhodamine Terminator Cycle Sequencing kit (PE Applied Biosystems, Foster City, Calif.) according to the manufacturer's instructions. Sequence reactions were analyzed using a Perkin Elmer/ABI Prism 310 automated sequencer.

TABLE 1
Dicot Codon Usage

| | | | | | % |
|---|---|---|---|---|---|
| Amino Acid | Codon | % Usage | Amino Acid | Codon | Usage |
| Alanine | GCU | 45.10 | Lysine | AAG | 55.14 |
| | GCA | 25.41 | | AAA | 44.86 |
| | GGC | 17.62 | Methionine | AUG | 100.00 |
| | GCG | 11.87 | Phenylalanine | UUC | 51.60 |
| Arginine | AGA | 32.71 | | UUU | 48.40 |
| | AGG | 23.18 | Proline | CCU | 37.73 |
| | CGU | 18.53 | | CCA | 34.72 |
| | CGA | 10.55 | | CCG | 14.94 |
| | CGG | 7.70 | | CCC | 12.61 |
| | CGC | 7.33 | Serine | UCU | 27.64 |
| Asparagine | AAC | 51.76 | | UCA | 19.32 |
| | AAU | 48.24 | | AGU | 15.84 |
| Aspartic Acid | GAU | 65.56 | | AGC | 13.98 |
| | GAG | 34.44 | | UCC | 13.50 |
| Cysteine | UGU | 56.16 | | UCG | 9.72 |
| | UGC | 43.84 | Threonine | ACU | 36.71 |
| Glutamic Acid | GAG | 51.01 | | ACA | 28.49 |
| | GAA | 48.99 | | ACC | 21.89 |
| Glutamine | CAA | 54.26 | | ACG | 12.91 |
| | CAG | 45.74 | Tryptophan | UGG | 100.00 |
| Glycine | GGA | 36.34 | Tyrosine | UAC | 52.04 |
| | GGU | 35.49 | | UAU | 47.96 |
| | GGC | 14.14 | Valine | GUU | 40.71 |
| | GGG | 14.03 | | GUG | 26.01 |
| Histidine | CAU | 57.39 | | GUC | 20.56 |
| | CAC | 42.61 | | GUA | 12.71 |
| Isoleucine | AUU | 43.44 | Stop | UAA | 41.50 |
| | AUC | 36.28 | | UGA | 40.73 |
| | AUA | 20.28 | | UAG | 17.77 |
| Leucine | UUG | 24.73 | | | |
| | CUC | 19.86 | | | |
| | CUU | 19.86 | | | |
| | UUA | 12.99 | | | |
| | CAG | 11.69 | | | |
| | CUA | 10.87 | | | |

TABLE 2
Monocot Codon Usage

| | | | | | % |
|---|---|---|---|---|---|
| Amino Acid | Codon | % Usage | Amino Acid | Codon | Usage |
| Alanine | GCC | 36.21 | Lysine | AAG | 79.42 |
| | GCG | 24.24 | | AAA | 20.58 |
| | GCU | 23.10 | Methionine | AUG | 100.00 |
| | GCA | 16.45 | | | |
| Arginine | AGG | 27.35 | | UUU | 29.32 |
| | CGC | 27.09 | Proline | CCA | 27.77 |
| | CGG | 15.18 | | CCG | 27.06 |
| | AGA | 12.94 | | CCC | 23.97 |
| | CGU | 11.41 | | CCU | 21.20 |
| | CGA | 6.03 | Serine | AGC | 24.00 |
| Asparagine | AAC | 68.33 | | UCC | 23.41 |
| | AAU | 31.67 | | UCU | 15.47 |
| Aspartic Acid | GAC | 62.72 | | UCG | 13.97 |
| | GAU | 37.28 | | UCA | 13.59 |
| Cysteine | UGC | 73.56 | | AGU | 9.57 |
| | UGU | 26.44 | Threonine | ACC | 40.33 |
| Glutamic Acid | GAG | 73.29 | | ACU | 21.27 |
| | GAA | 26.71 | | ACG | 20.39 |
| Glutamine | CAG | 58.31 | | ACA | 18.00 |
| | CAA | 41.69 | Tryptophan | UGG | 100.00 |
| Glycine | GGC | 41.40 | Tyrosine | UAC | 72.14 |
| | GGG | 20.28 | | UAU | 27.86 |
| | GGU | 20.25 | Valine | GUG | 37.35 |
| | GGA | 18.07 | | GUC | 32.71 |
| Histidine | CAC | 63.83 | | GUU | 22.11 |
| | CAU | 36.17 | | GUA | 7.83 |
| Isoleucine | AUC | 57.82 | Stop | UGA | 44.57 |
| | AUU | 28.52 | | UAG | 28.24 |
| | AUA | 136.5 | | UAA | 27.18 |
| Leucine | CUC | 27.01 | | | |
| | CUU | 27.01 | | | |
| | CUG | 23.80 | | | |
| | UUG | 11.90 | | | |
| | CUA | 6.23 | | | |
| | UUA | 4.05 | | | |

TABLE 3A

Dicot Oligonucleotides: Sense Strand

[SEQ ID NO:13]

AGCTAGATCCTTTTTATTTTTAATTTTCTTTCAAATACTTCCAG

[SEQ ID NO:14]

ATCCATGTCTGTTGTTGCTAACCCTTTGCATCCTTTGTTCGCTGCTGGAG
TTGAGGATATTGATCTCAGAGAAGCATTGG

[SEQ ID NO:15]

GTTCTACTGAGGTGAGAGAAATTGAGAGACTCATGGACGAAAAGTCAGTT
CTCGTTTTCAGAGGTCAACCACTCTCACAG

[SEQ ID NO:16]

GATCAACAGATTGCTTTTGCTAGGAATTTTGGACCTTTGGAGGGTGGATT
CATCAAAGTGAACCAGAGACCATCTAGGTT

[SEQ ID NO:17]

CAAATATGCTGAACTCGCTGATATCTCTAATGTTTCATTGGATGGTAAGG
TGGCACAAAGAGACGCTAGAGAAGTTGTGG

[SEQ ID NO:18]

GAAATTTTGCAAATCAATTGTGGCATTCTGATTCTTCATTCCAACAGCCA
GCAGCTAGATATTCTATGTTGTCAGCTGTT

[SEQ ID NO:19]

GTTGTGCCTCCTTCTGGAGGTGATACAGAATTTTGTGATATGAGGGCAGC
TTACGATGCTCTCCCAAGGGATTTGCAGTC

[SEQ ID NO:20]

TGAACTCGAGGGATTGAGAGCTGAACATTACGCTTTGAACTCAAGATTTC
TCTTGGGAGATACTGATTACTCAGAGGCAC

[SEQ ID NO:21]

AGAGAAACGCTATGCCTCCTGTTAACTGGCCTCTCGTTAGGACTCATGCT
GGTTCTGGTAGAAAGTTCTTGTTTATTGGA

[SEQ ID NO:22]

GCACATGCTTCACATGTTGAGGGTCTCCCTGTTGCTGAGGGAAGAATGTT
GCTCGCTGAATTGCTCGAACATGCTACTCA

[SEQ ID NO:23]

AAGAGAGTTTGTTTATAGACACAGATGGAATGTTGGTGACTTGGTTATGT
GGGATAATAGATGTGTGTTGCATAGAGGTA

[SEQ ID NO:24]

GGAGATATGATATTTCTGCTAGAAGGGAACTCAGAAGGGCTACTACTTTG
GATGACGCTGTTGTTTAGTGTGCTGGAG

TABLE 3B

Dicot Oligonucleotides: Nonsense Strand

[SEQ ID NO:25]

GAACAAAGGATGCAAAGGGTTAGCAACAACAGACATGGATCTGGAAGTAT
TTGAAAGAAAATTAAAAATAAAAAGGATCT

[SEQ ID NO:26]

TCGTCCATGAGTCTCTCAATTTCTCTCACCTCAGTAGAACCCAATGCTTC
TCTGAGATCAATATCCTCAACTCCAGCAGC

[SEQ ID NO:27]

CCAAAGGTCCAAAATTCCTAGCAAAAGCAATCTGTTGATCCTGTGAGAGT
GGTTGACCTCTGAAAACGAGAACTGACTTT

[SEQ ID NO:28]

CAATGAAACATTAGAGATATCAGCGAGTTCAGCATATTTGAACCTAGATG
GTCTCTGGTTCACTTTGATGAATCCACCCT

[SEQ ID NO:29]

AATGAAGAATCAGAATGCCACAATTGATTTGCAAAATTTCCCACAACTTC
TCTAGCGTCTCTTTGTGCCACCTTACCATC

[SEQ ID NO:30]

TATCACAAAATTCTGTATCACCTCCAGAAGGAGGCACAACAACAGCTGAC
AACATAGAATATCTAGCTGCTGGCTGTTGG

[SEQ ID NO:31]

GTTCAAGCGTAATGTTCAGCTCTCAATCCCTCGAGTTCAGACTGCAAAT
CCCTTGGGAGAGCATCGTAAGCTGCCCTCA

[SEQ ID NO:32]

CTAACGAGAGGCCAGTTAACAGGAGGCATAGCGTTTCTCTGTGCCTCTGA
GTAATCAGTATCTCCCAAGAGAAATCTTGA

[SEQ ID NO:33]

CCTCAGCAACAGGGAGACCCTCAACATGTGAAGCATGTGCTCCAATAAAC
AAGAACTTTCTACCAGAACCAGCATGAGTC

[SEQ ID NO:34]

GTCACCAACATTCCATCTGTGTCTATAAACAAACTCTCTTTGAGTAGCAT
GTTCGAGCAATTCAGCGAGCAACATTCTTC

[SEQ ID NO:35]

GCCCTTCTGAGTTCCCTTCTAGCAGAAATATCATATCTCCTACCTCTATG
CAACACACATCTATTATCCCACATAACCAA

[SEQ ID NO:36]

TCGACTCCAGCACACTAAACAACAGCGTCATCCAAAGTAGTA

TABLE 4A

Monocot oligonucleotides: Sense strand

[SEQ ID NO:37]

AGCTAGATCCTTTTTATTTTTAATTTTCTTTCAAATACTTCCAG

[SEQ ID NO:38]

ATCCATGTCCGTGGTGGCCAACCCACTCCACCCGCTCTTCGCGGCCGGCG
TGGAGGATATCGACCTCAGGGAGGCGCTGG

[SEQ ID NO:39]

GCAGCACCGAAGTGCGCGAAATCGAGAGGCTCATGOACGAGAAGAGCGTC
CTCGTCTTCCGCGGCCAACCACTCTCACAG

[SEQ ID NO:40]

GATCAACAGATTGCTTTTGCTAGGAATTTTGGACCTTTGGAGGGTGGATT
CATCAAGGTGAACCAGCGCCCGTCCAGGTT

[SEQ ID NO:41]

CAAGTACGCTGAACTGGCCGACATCAGCAACGTGTCCCTCGATGGGAAGG
TGGCCCAGAGGGACGCTAGGGAAGTTGTGG

[SEQ ID NO:42]

GCAACTTCGCCAACCAACTGTGGCACTCCGATAGCTCTTTCCAACAGCCA
GCAGCCAGGTACTCCATGCTGAGCGCCGTC

[SEQ ID NO:43]

GTCGTGCCACCATCCGGCGGTGACACCGAGTTCTGCGATATGCGCGCCGC
GTACGACGCCCTCCCGAGGGATCTGCAGAG

[SEQ ID NO:44]

CGAGCTGGAGGGCCTCCGCGCGGAGCACTACGCCCTCAACAGCAGGTTCC
TCCTGGGGGACACTGACTACTCCGAGGCCC

[SEQ ID NO:45]

AGAGGAACGCGATGCCACCAGTGAACTGGCCCCTCGTCCGCACCCACGCT
GGCAGCGGCCGCAAGTTCCTGTTCATCGGG

[SEQ ID NO:46]

GCCCATGCCTCCCATGTGGAGGGTCTCCCTGTCGCGGAGGGCCGCATGCT
CCTGGCCGAGCTCCTGGAGCACGCCACCCA

[SEQ ID NO:47]

ACGCGAGTTCGTCTACCGCCACAGGTGGAATGTCGGCGACCTCGTCATGT
GGGATAACCGCTGCGTGCTGCACCGCGGCA

[SEQ ID NO:48]

GGCGCTACGATATCAGCGCGCGCAGGGAACTCAGGCGCGCCACCACCCTC
GACGACGCGGTGGTGTAGTGTGCTGGAG

TABLE 4B

Monocot oligonucleotides: Nonsense strand

[SEQ ID NO:49]

GAAGAGCGGGTGGAGTGGGTTGGCCACCACGGACATGGATCTGGAAGTAT
TTGAAAGAAAATTAAAAATAAAAAGGATCT

[SEQ ID NO:50]

TCGTCCATGAGCCTCTCGATTTCGCGCACTTCGGTGCTGCCCAGCGCCTC
CCTGAGGTCGATATCCTCCACGCCGGCCGC

[SEQ ID NO:51]

CCAAAGGTCCAAAATTCCTAGCAAAAGCAATCTGTTGATCCTGTGAGAGT
GGTTGGCCGCGGAAGACGAGGACGCTCTTC

[SEQ ID NO:52]

GAGGGACACGTTGCTGATGTCGGCCAGTTCAGCGTACTTGAACCTGGACG
GGCGCTGGTTCACCTTGATGAATCCACCCT

[SEQ ID NO:53]

AAAGAGCTATCGGAGTGCCACAGTTGGTTGGCGAAGTTGCCCACAACTTC
CCTAGCGTCCCTCTGGGCCACCTTCCCATC

[SEQ ID NO:54]

TATCGCAGAACTCGGTGTCACCGCCGGATGGTGGCACGACGACGGCGCTC
AGCATGGAGTACCTGGCTGCTGGCTGTTGG

[SEQ ID NO:55]

GTTGAGGGCGTAGTGCTCCGCGCGGAGGCCCTCCAGCTCGCTCTGCAGAT
CCCTCGGGAGGGCGTCGTACGCGGCGCGCA

[SEQ ID NO:56]

CGGACGAGGGGCCAGTTCACTGGTGGCATCGCGTTCCTCTGGGCCTCGGA
GTAGTCAGTGTCCCCCAGGAGGAACCTGCT

[SEQ ID NO:57]

CCTCCGCGACAGGGAGACCCTCCACATGGGAGGCATGGGCCCCGATGAAC
AGGAACTTGCGGCCGCTGCCAGCGTGGGTG

[SEQ ID NO:53]

GTCGCCGACATTCCACCTGTGGCGGTAGACGAACTCGCGTTGGGTGGCGT
GCTCCAGGAGCTCGGCCAGGAGCATGCGGC

[SEQ ID NO:59]

GCGCGCCTGAGTTCCCTGCGCGCGCTGATATCGTAGCGCCTGCCGCGGTG
CAGCACGCAGCGGTTATCCCACATGACGAG

[SEQ ID NO:60]

TCGACTCCAGCACACTACACCACCGCGTCGTCGAGGGTGGTG

### Example 2

Construction of a Plant-Expressible SAD Gene.

For the generation of a complete and plant-competent SAD gene, the synthetic portions of the SAD gene contained in pUCRsynSAD were removed by first releasing the 5′ end of the synthetic sequence by digestion with XbaI and filling in the overhang with DNA polymerase I (Klenow large fragment) followed by digestion with KpnI. This fragment was ligated into the plasmid pProPClSV, a pUC19 plasmid containing an enhanced Peanut Chlorotic Streak Virus (PClSV) promoter derived from pKLP36 (described by Maiti and Shepherd, Biochem. Biophys. Res. Com., 244:440-444 (1998)) by cutting first with NcoI, treating with DNA polymerase I (Klenow large fragment) to fill in the generated overhang, and subsequently cutting with KpnI. This generated the plasmid pProPClSV-SAD within which the synthetic portion of the SAD gene, including the 5′ AMV leader and 3′ region coding for the Cys Ala Gly signature, is directly linked to the 3′ end of the PClSV promoter (FIG. 1). This plasmid served as the source for the PClSV-SAD construction for insertion into the binary vectors for final gene construction prior to introduction into Agrobacterium for plant transformation.

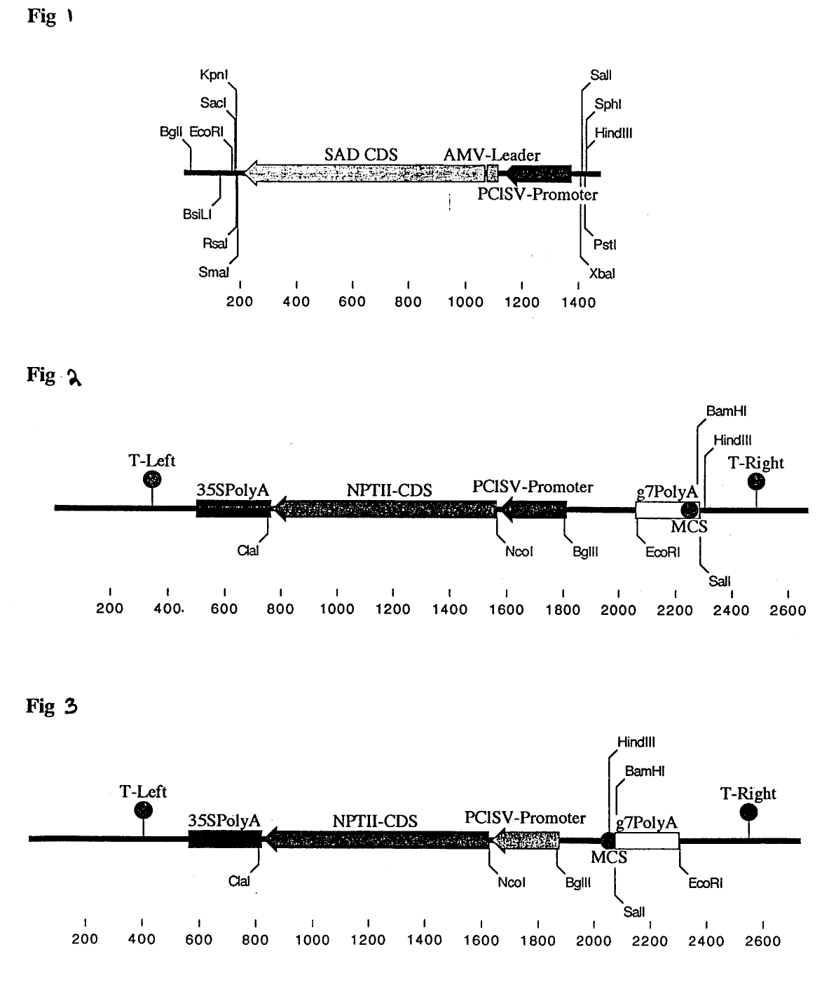

Two binary vectors were chosen for final SAD gene construction, pPZP211-PNPT-311g7 (FIG. 2) and pPZP211-PNPT-512g7 (FIG. 3). These two vectors are based on the pPZP family of vectors described by Hajdukiewicz et al., Plant Molec. Biol., 25:989-994 (1994) and are pPZP211 derivatives in which the neomycin phosphotransferase II (NPTII) gene for kanamycin resistance is driven by the PClSV promoter and a g7 polyA termination sequence is placed adjacent to a multicloning site (MCS, FIGS. 2 and 3). The only difference between these two vectors is the position of the MCS relative to the g7 polyA termination sequence. The g7 polyA termination sequence is the 3′ polyA termination signal from gene 7 within the octopine T-Left region of an octopine Agrobacterium tumefaciens Ti plasmid and was isolated as an EcoRI-SalI fragment from pAP2034 (Velten and Schell, Nucleic Acids, 13:6981-6998 (1985)).

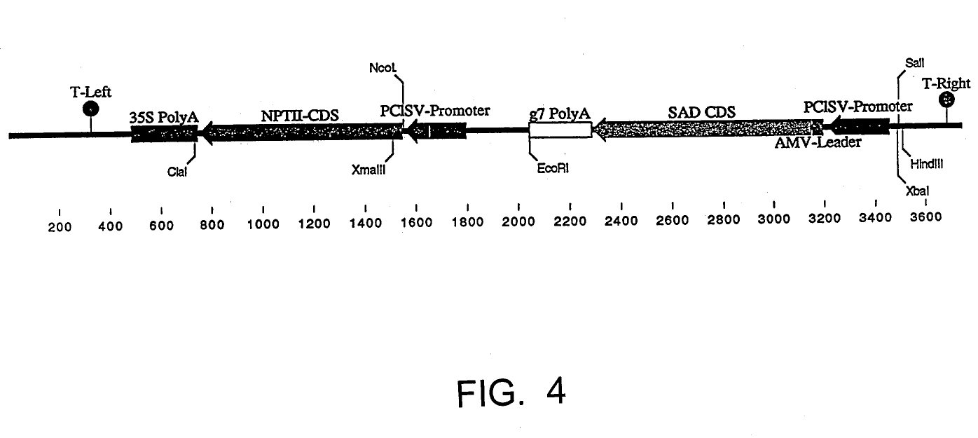

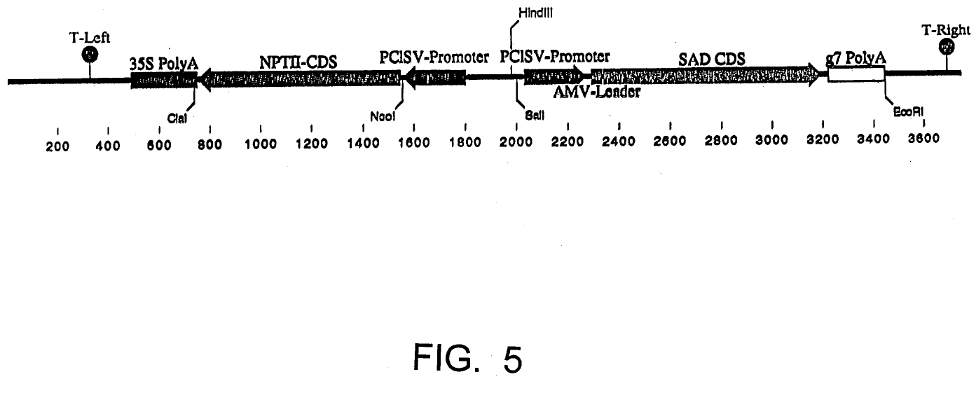

The complete SAD gene was constructed by removal of the PClSV-SAD sequence from pProPClSV-SAD as a HindIII-SmaI fragment and insertion into both pPZP211-PNPT-311g7 and pPZP211-PNPT-512g7 that were first cut with BamHI, treated with DNA polymerase I (Klenow large fragment) to fill in the overhanging sequence, and subsequently digested with HindIII. These reactions generated the two vectors, pPZP211-PNPT-311-SAD (FIG. 4) and pPZP211-PNPT-512-SAD (FIG. 5), that contained the full plant expressible SAD gene in one of two orientations with respect to the PClSV-NPTII-35SpolyA construct. This design for insertion of the SAD gene into plant genomes was implemented because of uncertainty as to the effect of having two PCLSV promoter sequences in the same plasmid on both transformation and effective transmission of the expressed trait. By putting the SAD gene in the vectors such that the PClSV promoters were inserted as both direct and inverted repeats, the possibility of a negative outcome could be avoided.

After construction, the SAD genes in each vector were sequenced as described above to ensure fidelity. This sequencing revealed that, in the construction of pProPClSV-SAD, an out-of-frame ATG codon was introduced into the 5′ untranslated leader sequence. The presence of this ATG codon could alter the translatability of the transcript that would be synthesized from the SAD gene and so was deleted by PCR mutagenesis to restore the normal AMV leader sequence. Following repair, the sequence was rechecked for fidelity. The original SAD gene containing the out-of-frame ATG was labelled SAD1 (since some transformation experiments had begun using this construct). The repaired SAD gene is referred to as SAD2 and is the only version of the gene used for integration of the SAD construct into the cotton genome.

### Example 3

Introduction of SAD2 into Cotton

The two binary vectors containing the SAD2 gene, pPZP211-PNPT-311-SAD2 and pPZP211-PNPT-512-SAD2, were individually introduced into the EHA 105 strain of Agrobacterium tumefaciens (Hood et al., Transgenic Research, 2:208-218 (1993)) by direct transformation as described by Walker-Peach and Velten, in Plant Molecular Biology Manual, section B1:1-19 (Gelvin, Shilperoort and Verma, eds., Kluwer Academic Publishers, Dordrecht, The Netherlands, 1994)). The constructs were subsequently introduced by Agrobacterium transfection into cotyledon explants from the cotton variety Coker 312 (Coker Seed Inc.). This was achieved by isolating sterile cotyledon tissue (derived from seedlings grown in culture from surface-sterilized seed as described by Trolinder and Gooden, Plant Cell Reports, 6:231-234 (1987)), generating explants (by use of a sterile hole punch), and submerging the explants in a 48-hour-old culture of EHA 105, containing the appropriate construct, grown at 28° C. The explants were then transferred onto 2MST medium (MS medium+0.2 mg/L 2,4-D and 0.1 mg/L kinetin) subsequent to removal of excess EHA 105. The infected cotyledon tissues were incubated on the 2MST medium for 2 days at 28° C. prior to transfer to T1+KCL medium (MS medium+0.1 mg/L 2,4-D and 0.1 mg/L kinetin+50 mg/L kanamycin sulphate and 250 mg/L Cefotaxime). Once healthy callus tissue was formed, it was placed on fresh T1+KCL (with 0.05 mg/L 2,4-D) for a second round of selection. After six weeks, somatic embryos were generated from the surviving callus, and mature transgenic cotton plants were produced as described by Trolinder and Goodin, Plant Cell Reports, 6:231-234 (1987).

A total of 111 kanamycin-resistant cotton seedlings were generated (44 were generated in the pPZP211-PNPT-311-SAD2 transformations, and 67 in the pPZP211-PNPT-512-SAD2 transformations). Each plant was analyzed for the presence of the SAD synthetic coding sequence and the NPTII coding sequence by PCR to ensure the integrity of the inserted DNA. The PCR was performed as described above. The primers used for this analysis were GGAGTTGAGGATATTGATCTCAGAGAAGCATTG [SEQ ID NO:9] and GCGATCTGCTGATCCTGACTC [SEQ ID NO:10] for the SAD coding region and CGTCAAGAAGGCGATAGAAGG [SEQ ID NO:11] and GCTATGACTGGGCACAACAGAC [SEQ ID NO:12] for the NPTII coding region. Of the 44 pPZP211-PNPT-311-SAD seedlings that survived kanamycin treatment, 2 were shown to be negative by the PCR testing. Of the 67 pPZP211-PNPT-512-SAD seedlings that survived kanamycin treatment, 14 were negative in the PCR tests.

The remaining 95 plants were grown in pots in the greenhouse until at least the squaring stage (approximately 18″ tall) and were then sprayed with 2,4-D amine (United Agri Products, Greeley, Colo.) at 1 lb/acre acid equivalents. This is twice the normal field rate for 2,4-D applications. Of the 95 plants, 22 survived this treatment with little or no herbicide damage being evident. Eleven of these plants contained the pPZP211-PNPT-311-SAD2 construct, and 11 contained the pPZP211-PNPT-512-SAD2 construct. Each plant was grown to maturity in the greenhouse.

Of the 22 transgenic plants, only 7 produced seed. The remaining plants were apparently infertile. Presumably this infertility was an effect of the regeneration procedure, which is common for cotton. Of the seven fertile plants, 3 contained the pPZP211-PNPT-311-SAD2 construct, and 4 contained the pPZP211-PNPT-311-SAD2 construct.

To verify that the inserted synthetic SAD genes were inheritable and to gain an indication of the number of gene insertions, seeds from the seven fertile SAD transgenic cotton plants were planted into hydroponic rock wool slabs (Hummert, St. Louis, Mo.) that had been saturated with Peters Professional water-soluble fertilizer (5-11-26 HYDRO-SOL, supplemented with calcium nitrate and magnesium sulfate to provide a complete nutrient compliment; Hummert, St. Louis, Mo.). The hydroponic rock wool slabs were placed on benches in a greenhouse, and nutrients were maintained at optimal levels using a non-recycling hydroponic watering system. Plants were grown under greenhouse conditions (28° C.±5° C. air temperature) for 24 days. At this point, the plants were removed to a spray hood, sprayed with 2,4-D amine at the normal field rate of 1/2 lb/acre acid equivalents, maintained in the hood for 24 hours to allow the 2,4-D to volatilize, and then placed back in the greenhouse. The effect of the treatment was evaluated visually after 10-14 days, and the results are presented in Table 5 below.

TABLE 5

| Transgenic | Number of | No | Some | Severe | Ratio |
|---|---|---|---|---|---|
| Line | plants | Damage | symptoms | damage | “Res”/Sens |
| 311-22-1-4 | 43 | 16 | 16 | 11 | 3:1 |
| 311-22-1-5 | 47 | 6 | 18 | 23 | 1:1 |
| 311-22-1-8 | 56 | 14 | 28 | 14 | 3:1 |
| 512-21-1-5 | 56 | 8 | 34 | 14 | 3:1 |
| 512-21-4-3 | 30 | 7 | 20 | 3 | 9:1 |
| 512-21-4-5 | 41 | 5 | 24 | 12 | 2:1 |
| 512-24-4-4 | 63 | 7 | 21 | 35 | 1:1 |
| 2,4-D res | 55 | 7 | 33 | 15 | 3:1 |
| control | | | | | |
| 2,4-D sens | 45 | 0 | 20 | 25 | 1:1 |
| control | | | | | |

2,4-D res control = transgenic 2,4-D resistant cotton containing the naturally-occurring tfdA gene construct. (Bayley et al., Theoretical Applied Genetics, 83:645-649 (1992))
2,4-D sens control = Coker 312 (not transgenic) regenerated from somatic embryos in the same manner as those containing the SAD constructs
Some symptoms = some leaf wilt and minor leaf dessication
Severe damage = all leaves wilted and desiccation damage readily evident

The ratio “Res”/Sens was calculated as the number of plants that showed some resistance to 2,4-D treatment during the experiment divided by the combined number of plants that showed severe damage or death. The negative control of Coker 312 that had been regenerated from tissue culture did show some signs of resistance, so these ratios are not to be considered as definitive measures of Mendelian inheritance of the SAD gene. Nevertheless, all of the negative control plants did show 2,4-D-induced damage, whereas all of the transgenic lines that contain the SAD gene had individuals that exhibited no damage at all.

Five plants from each of the 7 lines that exhibited no damage when treated with 2,4-D 24 days after germination were chosen, and individual newly-formed leaf samples, one per plant, were taken for PCR testing, performed as described above. Each plant tested positive for the SAD construct by PCR. These plants were grown for a further 14 days and then resprayed with 2,4-D amine at the normal field rate of 1/2 lb/acre acid equivalents. All 35 plants exhibited no damage following this treatment, whereas all negative controls did not survive this spray event. The 35 plants were grown to maturity, and seeds were collected.

The content of each of the references referred to hereinabove, including publications, patents, and published applications, are incorporated by reference herein.

We claim:

1. A DNA molecule comprising a synthetic DNA sequence,
the synthetic DNA sequence encoding an enzyme that degrades 2,4-dichlorophenoxyacetic acid to dichlorophenol, and
the synthetic DNA sequence comprising a natural microbial sequence that encodes the enzyme in which at least a plurality of the codons of the natural microbial sequence have been replaced by codons more preferred by a plant.

2. The DNA molecule of claim 1 wherein all of the codons of the natural microbial sequence which are least preferred by the plant have been replaced by codons more preferred by the plant.

3. The DNA molecule of claim 1 wherein at least 50% of the codons of the natural microbial sequence have been replaced by codons more preferred by the plant.

4. The DNA molecule of claim 3 wherein at least 80% of the codons of the natural microbial sequence have been replaced by codons more preferred by the plant.

5. The DNA molecule of any one of claims 1-4 wherein the natural microbial sequence is a natural bacterial sequence.

6. The DNA molecule of any one of claims 1-4 wherein the codons of the natural microbial sequence have been replaced by codons more preferred by a dicotyledonous plant.

7. The DNA molecule of claim 6 comprising the nucleotide sequence of SEQ ID NO:2.

8. The DNA molecule of any one of claims 1-4 wherein the codons of the natural microbial sequence have been replaced by codons more preferred by a monocotyledonous plant.

9. The DNA molecule of claim 8 comprising the nucleotide sequence of SEQ ID NO:3.

10. A DNA construct comprising a synthetic DNA sequence,
the synthetic DNA sequence being operatively linked to plant gene expression control sequences,
the synthetic DNA sequence encoding an enzyme that degrades 2,4-dichlorophenoxyacetic acid to dichlorophenol, and
the synthetic DNA sequence comprising a natural microbial sequence that encodes the enzyme in which at least a plurality of the codons of the natural microbial sequence have been replaced by codons more preferred by a plant.

11. The DNA construct of claim 10 wherein all of the codons of the natural microbial sequence which are least preferred by the plant have been replaced by codons more preferred by the plant.

12. The DNA construct of claim 10 wherein at least 50% of the codons of the natural microbial sequence have been replaced by codons more preferred by the plant.

13. The DNA construct of claim 12 wherein at least 80% of the codons of the natural microbial sequence have been replaced by codons more preferred by the plant.

14. The DNA construct of any one of claims 10-13 wherein the natural microbial sequence is a natural bacterial sequence.

15. The DNA construct of any one of claims 10-13 wherein the codons of the natural microbial sequence have been replaced by codons more preferred by a dicotyledonous plant.

16. The DNA construct of claim 15 comprising the nucleotide sequence of SEQ ID NO:2.

17. The DNA construct of claim 15 comprising the nucleotide sequence of SEQ ID NO:4.

18. The DNA construct of any one of claims 10-13 wherein the codons of the natural microbial sequence have been replaced by codons more preferred by a monocotyledonous plant.

19. The DNA construct of claim 18 comprising the nucleotide sequence of SEQ ID NO:3.

20. The DNA construct of claim 18 comprising the nucleotide sequence of SEQ ID NO:5.

21. The DNA construct of claim 10 which is a vector.

22. The DNA construct of claim 10 which is a plasmid.

23. The DNA construct of claim 10 which is pProPClSV-SAD.

24. The DNA construct of claim 10 which is pPZP211-PNPT-311-SAD.

25. The DNA construct of claim 10 which is pPZP211-PNPT-512-SAD.

26. The DNA construct of claim 10 wherein the expression control sequences comprise a Peanut Chlorotic Streak Virus promoter.

27. A transgenic plant or part of a plant comprising a synthetic DNA sequence,
the synthetic DNA sequence being operatively linked to plant gene expression control sequences,
the synthetic DNA sequence encoding an enzyme that degrades 2,4-dichlorophenoxyacetic acid to dichlorophenol, and
the synthetic DNA sequence comprising a natural microbial sequence that encodes the enzyme in which at least a plurality of the codons of the natural microbial sequence have been replaced by codons more preferred by a plant.

28. The plant or plant part of claim 27 wherein the expression control sequences comprise a Peanut Chlorotic Streak Virus promoter.

29. The plant or plant part of claim 27 wherein all of the codons of the natural microbial sequence which are least preferred by the plant have been replaced by codons more preferred by the plant.

30. The plant or plant part of claim 27 wherein at least 50% of the codons of the natural microbial sequence have been replaced by codons more preferred by the plant.

31. The plant or plant part of claim 30 wherein at least 80% of the codons of the natural microbial sequence have been replaced by codons more preferred by the plant.

32. The plant or plant part of any one of claims 27-31 wherein the natural microbial sequence is a natural bacterial sequence.

33. The plant or plant part of claim 27 which is a dicotyledonous plant or plant part and the codons of the natural microbial sequence have been replaced by codons more preferred by a dicotyledonous plant.

34. The plant or plant part of claim 33 wherein the one or more cells comprise the nucleotide sequence of SEQ ID NO:2.

35. The plant or plant part of claim 33 wherein the one or more cells comprise the nucleotide sequence of SEQ ID NO:4.

36. The plant or plant part of claim 27 which is a monocotyledonous plant or plant part and the codons of the natural microbial sequence have been replaced by codons more preferred by a monocotyledonous plant.

37. The plant or plant part of claim 36 wherein the one or more cells comprise the nucleotide sequence of SEQ ID NO:3.

38. The plant or plant part of claim 36 wherein the one or more cells comprise the nucleotide sequence of SEQ ID NO:5.

39. The plant or plant part of claim 27 which is a cotton plant or plant part.

40. The plant part of claim 27 which is a seed.

41. The plant part of claim 27 which is a fruit.

42. A method of controlling weeds in a field containing a transgenic plant,
the method comprising applying an amount of an auxin herbicide to the field effective to control the weeds in the field,
the transgenic plant being tolerant to the auxin herbicide as a result of comprising a synthetic DNA sequence,
the synthetic DNA sequence being operatively linked to plant gene expression control sequences,
the synthetic DNA sequence encoding an enzyme that degrades 2,4-dichlorophenoxyacetic acid to dichlorophenol, and
the synthetic DNA sequence comprising a natural microbial sequence that encodes the enzyme in which at least a plurality of the codons of the natural microbial sequence have been replaced by codons more preferred by the plant.

43. The method of claim 42 wherein the auxin herbicide is 2,4-dichlorophenoxyacetic acid (2,4-D) or 2,4-D amine.

44. The method of claim 42 wherein the plant is a monocotyledonous plant.

45. The method of claim 42 wherein the plant is a dicotyledonous plant.

46. The method of claim 42 wherein the plant is a cotton plant.

47. A method of selecting transformed plant cells comprising:
providing a population of plant cells;
transforming at least some of the plant cells in the population with the DNA construct of claim 10; and
selecting the transformed plant cells by culturing the resulting population of plant cells in a culture medium containing an auxin herbicide at a concentration selected so that transformed plant cells proliferate and untransformed plant cells do not proliferate.

48. The method of claim 47 wherein the auxin herbicide is 2,4-dichlorophenoxyacetic acid (2,4-D) or 2,4-D amine.

49. A method of selecting transformed plants comprising:
providing a population of plants which may comprise one or more plants comprising a DNA construct according to claim 10; and
selecting transformed plants by applying an amount of an auxin herbicide to the population of plants, the amount of herbicide being selected so that transformed plants grow and growth of untransformed plants is inhibited.

50. The method of claim 49 wherein the auxin herbicide is 2,4-dichlorophenoxyacetic acid (2,4-D) or 2,4-D amine.


SEQUENCE LISTING (60 sequences)

SEQ ID NO: 1 - protein, 287 aa, Alcaligenes eutrophus
MSVVANPLHPLFAAGVEDIDLREALGSTEVREIERLMDEKSVLVFRGQPLSQDQQIAFARNFGPLEGGFIKVNQRPSRFKYAELADISNVSLDGKVAQRDAREVVGNFANQLWHSDSSFQQPAARYSMLSAVVVPPSGGDTEFCDMRAAYDALPRDLQSELEGLRAEHYALNSRFLLGDTDYSEAQRNAMPPVNWPLVRTHAGSGRKFLFIGAHASHVEGLPVAEGRMLLAELLEHATQREFVYRHRWNVGDLVMWDNRCVLHRGRRYDISARRELRRATTLDDAVV

SEQ ID NO: 2 - DNA, 864 bp, Artificial sequence
  exon (1)..(864)
  misc_feature (1)..(864): Dicot ORF for degradation of 2,4-D
atgtctgttgttgctaaccctttgcatcctttgttcgctgctggagttgaggatattgatctcagagaagcattgggttctactgaggtgagagaaattgagagactcatggacgaaaagtcagttctcgttttcagaggtcaaccactctcacaggatcaacagattgcttttgctaggaattttggacctttggagggtggattcatcaaagtgaaccagagaccatctaggttcaaatatgctgaactcgctgatatctctaatgtttcattggatggtaaggtggcacaaagagacgctagagaagttgtgggaaattttgcaaatcaattgtggcattctgattcttcattccaacagccagcagctagatattctatgttgtcagctgttgttgtgcctccttctggaggtgatacagaattttgtgatatgagggcagcttacgatgctctcccaagggatttgcagtctgaactcgagggattgagagctgaacattacgctttgaactcaagatttctcttgggagatactgattactcagaggcacagagaaacgctatgcctcctgttaactggcctctcgttaggactcatgctggttctggtagaaagttcttgtttattggagcacatgcttcacatgttgagggtctccctgttgctgagggaagaatgttgctcgctgaattgctcgaacatgctactcaaagagagtttgtttatagacacagatggaatgttggtgacttggttatgtgggataatagatgtgtgttgcatagaggtaggagatatgatatttctgctagaagggaactcagaagggctactactttggatgacgctgttgtttag

SEQ ID NO: 3 - DNA, 864 bp, Artificial sequence
  exon (1)..(864)
  misc_feature (1)..(864): Monocot ORF for degradation of 2,4-D
atgtccgtggtggccaacccactccacccgctcttcgcggccggcgtggaggatatcgacctcagggaggcgctgggcagcaccgaagtgcgcgaaatcgagaggctcatggacgagaagagcgtcctcgtcttccgcggccaaccactctcacaggatcaacagattgcttttgctaggaattttggacctttggagggtggattcatcaaggtgaaccagcgcccgtccaggttcaagtacgctgaactggccgacatcagcaacgtgtccctcgatgggaaggtggcccagagggacgctagggaagttgtgggcaacttcgccaaccaactgtggcactccgatagctctttccaacagccagcagccaggtactccatgctgagcgccgtcgtcgtgccaccatccggcggtgacaccgagttctgcgatatgcgcgccgcgtacgacgccctcccgagggatctgcagagcgagctggagggcctccgcgcggagcactacgccctcaacagcaggttcctcctgggggacactgactactccgaggcccagaggaacgcgatgccaccagtgaactggcccctcgtccgcacccacgctggcagcggccgcaagttcctgttcatcggggcccatgcctcccatgtggagggtctccctgtcgcggagggccgcatgctcctggccgagctcctggagcacgccacccaacgcgagttcgtctaccgccacaggtggaatgtcggcgacctcgtcatgtgggataaccgctgcgtgctgcaccgcggcaggcgctacgatatcagcgcgcgcagggaactcaggcgcgccaccaccctcgacgacgcggtggtgtag

SEQ ID NO: 4 - DNA, 918 bp, Artificial sequence
  exon (45)..(908)
  misc_feature (1)..(918): Dicot gene for degradation of 2,4-D
agatcctttttatttttaattttctttcaaatacttccagatccatgtctgttgttgctaaccctttgcatcctttgttcgctgctggagttgaggatattgatctcagagaagcattgggttctactgaggtgagagaaattgagagactcatggacgaaaagtcagttctcgttttcagaggtcaaccactctcacaggatcaacagattgcttttgctaggaattttggacctttggagggtggattcatcaaagtgaaccagagaccatctaggttcaaatatgctgaactcgctgatatctctaatgtttcattggatggtaaggtggcacaaagagacgctagagaagttgtgggaaattttgcaaatcaattgtggcattctgattcttcattccaacagccagcagctagatattctatgttgtcagctgttgttgtgcctccttctggaggtgatacagaattttgtgatatgagggcagcttacgatgctctcccaagggatttgcagtctgaactcgagggattgagagctgaacattacgctttgaactcaagatttctcttgggagatactgattactcagaggcacagagaaacgctatgcctcctgttaactggcctctcgttaggactcatgctggttctggtagaaagttcttgtttattggagcacatgcttcacatgttgagggtctccctgttgctgagggaagaatgttgctcgctgaattgctcgaacatgctactcaaagagagtttgtttatagacacagatggaatgttggtgacttggttatgtgggataatagatgtgtgttgcatagaggtaggagatatgatatttctgctagaagggaactcagaagggctactactttggatgacgctgttgtttagtgtgctggag

SEQ ID NO: 5 - DNA, 918 bp, Artificial sequence
  exon (45)..(905)
  misc_feature (1)..(918): Monocot gene for degradation of 2,4-D
agatcctttttatttttaattttctttcaaatacttccagatccatgtccgtggtggccaacccactccacccgctcttcgcggccggcgtggaggatatcgacctcagggaggcgctgggcagcaccgaagtgcgcgaaatcgagaggctcatggacgagaagagcgtcctcgtcttccgcggccaaccactctcacaggatcaacagattgcttttgctaggaattttggacctttggagggtggattcatcaaggtgaaccagcgcccgtccaggttcaagtacgctgaactggccgacatcagcaacgtgtccctcgatgggaaggtggcccagagggacgctagggaagttgtgggcaacttcgccaaccaactgtggcactccgatagctctttccaacagccagcagccaggtactccatgctgagcgccgtcgtcgtgccaccatccggcggtgacaccgagttctgcgatatgcgcgccgcgtacgacgccctcccgagggatctgcagagcgagctggagggcctccgcgcggagcactacgccctcaacagcaggttcctcctgggggacactgactactccgaggcccagaggaacgcgatgccaccagtgaactggcccctcgtccgcacccacgctggcagcggccgcaagttcctgttcatcggggcccatgcctcccatgtggagggtctccctgtcgcggagggccgcatgctcctggccgagctcctggagcacgccacccaacgcgagttcgtctaccgccacaggtggaatgtcggcgacctcgtcatgtgggataaccgctgcgtgctgcaccgcggcaggcgctacgatatcagcgcgcgcagggaactcaggcgcgccaccaccctcgacgacgcggtggtgtagtgtgctggag

SEQ ID NO: 6 - DNA, 28 bp, Artificial sequence
  misc_feature (1)..(28): primer
agatcctttttatttttaattttctttc

SEQ ID NO: 7 - DNA, 25 bp, Artificial sequence
  misc_feature (1)..(25): primer
ctccagcacactaaacaacagcgtc

SEQ ID NO: 8 - DNA, 20 bp, Artificial sequence
  misc_feature (1)..(20): primer
ctccagcacactacaccacc

SEQ ID NO: 9 - DNA, 33 bp, Artificial sequence
  misc_feature (1)..(33): primer
ggagttgaggatattgatctcagagaagcattg

SEQ ID NO: 10 - DNA, 21 bp, Artificial sequence
  misc_feature (1)..(21): primer
gcgatctgctgatcctgactc

SEQ ID NO: 11 - DNA, 21 bp, Artificial sequence
  misc_feature (1)..(21): primer
cgtcaagaaggcgatagaagg

SEQ ID NO: 12 - DNA, 22 bp, Artificial sequence
  misc_feature (1)..(22): primer
gctatgactgggcacaacagac

SEQ ID NO: 13 - DNA, 44 bp, Artificial sequence
  misc_feature (1)..(44): gene fragment
agctagatcctttttatttttaattttctttcaaatacttccag

SEQ ID NO: 14 - DNA, 80 bp, Artificial sequence
  misc_feature (1)..(80): gene fragment
atccatgtctgttgttgctaaccctttgcatcctttgttcgctgctggagttgaggatattgatctcagagaagcattgg

SEQ ID NO: 15 - DNA, 80 bp, Artificial sequence
  misc_feature (1)..(80): gene fragment
gttctactgaggtgagagaaattgagagactcatggacgaaaagtcagttctcgttttcagaggtcaaccactctcacag

SEQ ID NO: 16 - DNA, 80 bp, Artificial sequence
  misc_feature (1)..(80): gene fragment
gatcaacagattgcttttgctaggaattttggacctttggagggtggattcatcaaagtgaaccagagaccatctaggtt

SEQ ID NO: 17 - DNA, 80 bp, Artificial sequence
  misc_feature (1)..(80): gene fragment
caaatatgctgaactcgctgatatctctaatgtttcattggatggtaaggtggcacaaagagacgctagagaagttgtgg

SEQ ID NO: 18 - DNA, 80 bp, Artificial sequence
  misc_feature (1)..(80): gene fragment
gaaattttgcaaatcaattgtggcattctgattcttcattccaacagccagcagctagatattctatgttgtcagctgtt

SEQ ID NO: 19 - DNA, 80 bp, Artificial sequence
  misc_feature (1)..(80): gene fragment
gttgtgcctccttctggaggtgatacagaattttgtgatatgagggcagcttacgatgctctcccaagggatttgcagtc

SEQ ID NO: 20 - DNA, 80 bp, Artificial sequence
  misc_feature (1)..(80): gene fragment
tgaactcgagggattgagagctgaacattacgctttgaactcaagatttctcttgggagatactgattactcagaggcac

SEQ ID NO: 21 - DNA, 80 bp, Artificial sequence
  misc_feature (1)..(80): gene fragment
agagaaacgctatgcctcctgttaactggcctctcgttaggactcatgctggttctggtagaaagttcttgtttattgga

SEQ ID NO: 22 - DNA, 80 bp, Artificial sequence
  misc_feature (1)..(80): gene fragment
gcacatgcttcacatgttgagggtctccctgttgctgagggaagaatgttgctcgctgaattgctcgaacatgctactca

SEQ ID NO: 23 - DNA, 80 bp, Artificial sequence
  misc_feature (1)..(80): gene fragment
aagagagtttgtttatagacacagatggaatgttggtgacttggttatgtgggataatagatgtgtgttgcatagaggta

SEQ ID NO: 24 - DNA, 78 bp, Artificial sequence
  misc_feature (1)..(78): gene fragment
ggagatatgatatttctgctagaagggaactcagaagggctactactttggatgacgctgttgtttagtgtgctggag

SEQ ID NO: 25 - DNA, 80 bp, Artificial sequence
  misc_feature (1)..(80): gene fragment
gaacaaaggatgcaaagggttagcaacaacagacatggatctggaagtatttgaaagaaaattaaaaataaaaaggatct

SEQ ID NO: 26 - DNA, 80 bp, Artificial sequence
  misc_feature (1)..(80): gene fragment
tcgtccatgagtctctcaatttctctcacctcagtagaacccaatgcttctctgagatcaatatcctcaactccagcagc

SEQ ID NO: 27 - DNA, 80 bp, Artificial sequence
  misc_feature (1)..(80): gene fragment
ccaaaggtccaaaattcctagcaaaagcaatctgttgatcctgtgagagtggttgacctctgaaaacgagaactgacttt

SEQ ID NO: 28 - DNA, 80 bp, Artificial sequence
  misc_feature (1)..(80): gene fragment
caatgaaacattagagatatcagcgagttcagcatatttgaacctagatggtctctggttcactttgatgaatccaccct

SEQ ID NO: 29 - DNA, 80 bp, Artificial sequence
  misc_feature (1)..(80): gene fragment
aatgaagaatcagaatgccacaattgatttgcaaaatttcccacaacttctctagcgtctctttgtgccaccttaccatc

SEQ ID NO: 30 - DNA, 80 bp, Artificial sequence
  misc_feature (1)..(80): gene fragment
tatcacaaaattctgtatcacctccagaaggaggcacaacaacagctgacaacatagaatatctagctgctggctgttgg

SEQ ID NO: 31 - DNA, 80 bp, Artificial sequence
  misc_feature (1)..(80): gene fragment
gttcaaagcgtaatgttcagctctcaatccctcgagttcagactgcaaatcccttgggagagcatcgtaagctgccctca

SEQ ID NO: 32 - DNA, 80 bp, Artificial sequence
  misc_feature (1)..(80): gene fragment
ctaacgagaggccagttaacaggaggcatagcgtttctctgtgcctctgagtaatcagtatctcccaagagaaatcttga

SEQ ID NO: 33 - DNA, 80 bp, Artificial sequence
  misc_feature (1)..(80): gene fragment
cctcagcaacagggagaccctcaacatgtgaagcatgtgctccaataaacaagaactttctaccagaaccagcatgagtc

SEQ ID NO: 34 - DNA, 80 bp, Artificial sequence
  misc_feature (1)..(80): gene fragment
gtcaccaacattccatctgtgtctataaacaaactctctttgagtagcatgttcgagcaattcagcgagcaacattcttc

SEQ ID NO: 35 - DNA, 80 bp, Artificial sequence
  misc_feature (1)..(80): gene fragment
gcccttctgagttcccttctagcagaaatatcatatctcctacctctatgcaacacacatctattatcccacataaccaa

SEQ ID NO: 36 - DNA, 42 bp, Artificial sequence
  misc_feature (1)..(42): gene fragment
tcgactccagcacactaaacaacagcgtcatccaaagtagta

SEQ ID NO: 37 - DNA, 44 bp, Artificial sequence; identical to SEQ ID NO: 13
  misc_feature (1)..(44): gene fragment

SEQ ID NO: 38 - DNA, 80 bp, Artificial sequence
  misc_feature (1)..(80): gene fragment
atccatgtccgtggtggccaacccactccacccgctcttcgcggccggcgtggaggatatcgacctcagggaggcgctgg

SEQ ID NO: 39 - DNA, 80 bp, Artificial sequence
  misc_feature (1)..(80): gene fragment
gcagcaccgaagtgcgcgaaatcgagaggctcatggacgagaagagcgtcctcgtcttccgcggccaaccactctcacag

SEQ ID NO: 40 - DNA, 80 bp, Artificial sequence
  misc_feature (1)..(80): gene fragment
gatcaacagattgcttttgctaggaattttggacctttggagggtggattcatcaaggtgaaccagcgcccgtccaggtt

SEQ ID NO: 41 - DNA, 80 bp, Artificial sequence
  misc_feature (1)..(80): gene fragment
caagtacgctgaactggccgacatcagcaacgtgtccctcgatgggaaggtggcccagagggacgctagggaagttgtgg

SEQ ID NO: 42 - DNA, 80 bp, Artificial sequence
  misc_feature (1)..(80): gene fragment
gcaacttcgccaaccaactgtggcactccgatagctctttccaacagccagcagccaggtactccatgctgagcgccgtc

SEQ ID NO: 43 - DNA, 80 bp, Artificial sequence
  misc_feature (1)..(80): gene fragment
gtcgtgccaccatccggcggtgacaccgagttctgcgatatgcgcgccgcgtacgacgccctcccgagggatctgcagag

SEQ ID NO: 44 - DNA, 80 bp, Artificial sequence
  misc_feature (1)..(80): gene fragment
cgagctggagggcctccgcgcggagcactacgccctcaacagcaggttcctcctgggggacactgactactccgaggccc

SEQ ID NO: 45 - DNA, 80 bp, Artificial sequence
  misc_feature (1)..(80): gene fragment
agaggaacgcgatgccaccagtgaactggcccctcgtccgcacccacgctggcagcggccgcaagttcctgttcatcggg

SEQ ID NO: 46 - DNA, 80 bp, Artificial sequence
  misc_feature (1)..(80): gene fragment
gcccatgcctcccatgtggagggtctccctgtcgcggagggccgcatgctcctggccgagctcctggagcacgccaccca

SEQ ID NO: 47 - DNA, 80 bp, Artificial sequence
  misc_feature (1)..(80): gene fragment
acgcgagttcgtctaccgccacaggtggaatgtcggcgacctcgtcatgtgggataaccgctgcgtgctgcaccgcggca

SEQ ID NO: 48 - DNA, 78 bp, Artificial sequence
  misc_feature (1)..(78): gene fragment
ggcgctacgatatcagcgcgcgcagggaactcaggcgcgccaccaccctcgacgacgcggtggtgtagtgtgctggag

SEQ ID NO: 49 - DNA, 80 bp, Artificial sequence
  misc_feature (1)..(80): gene fragment
gaagagcgggtggagtgggttggccaccacggacatggatctggaagtatttgaaagaaaattaaaaataaaaaggatct

SEQ ID NO: 50 - DNA, 80 bp, Artificial sequence
  misc_feature (1)..(80): gene fragment
tcgtccatgagcctctcgatttcgcgcacttcggtgctgcccagcgcctccctgaggtcgatatcctccacgccggccgc

SEQ ID NO: 51 - DNA, 80 bp, Artificial sequence
  misc_feature (1)..(80): gene fragment
ccaaaggtccaaaattcctagcaaaagcaatctgttgatcctgtgagagtggttggccgcggaagacgaggacgctcttc

SEQ ID NO: 52 - DNA, 80 bp, Artificial sequence
  misc_feature (1)..(80): gene fragment
gagggacacgttgctgatgtcggccagttcagcgtacttgaacctggacgggcgctggttcaccttgatgaatccaccct

SEQ ID NO: 53 - DNA, 80 bp, Artificial sequence
  misc_feature (1)..(80): gene fragment
aaagagctatcggagtgccacagttggttggcgaagttgcccacaacttccctagcgtccctctgggccaccttcccatc

SEQ ID NO: 54 - DNA, 80 bp, Artificial sequence
  misc_feature (1)..(80): gene fragment
tatcgcagaactcggtgtcaccgccggatggtggcacgacgacggcgctcagcatggagtacctggctgctggctgttgg

SEQ ID NO: 55 - DNA, 80 bp, Artificial sequence
  misc_feature (1)..(80): gene fragment
gttgagggcgtagtgctccgcgcggaggccctccagctcgctctgcagatccctcgggagggcgtcgtacgcggcgcgca

SEQ ID NO: 56 - DNA, 80 bp, Artificial sequence
  misc_feature (1)..(80): gene fragment
cggacgaggggccagttcactggtggcatcgcgttcctctgggcctcggagtagtcagtgtcccccaggaggaacctgct

SEQ ID NO: 57 - DNA, 80 bp, Artificial sequence
  misc_feature (1)..(80): gene fragment
cctccgcgacagggagaccctccacatgggaggcatgggccccgatgaacaggaacttgcggccgctgccagcgtgggtg

SEQ ID NO: 58 - DNA, 80 bp, Artificial sequence
  misc_feature (1)..(80): gene fragment
gtcgccgacattccacctgtggcggtagacgaactcgcgttgggtggcgtgctccaggagctcggccaggagcatgcggc

SEQ ID NO: 59 - DNA, 80 bp, Artificial sequence
  misc_feature (1)..(80): gene fragment
gcgcgcctgagttccctgcgcgcgctgatatcgtagcgcctgccgcggtgcagcacgcagcggttatcccacatgacgag

SEQ ID NO: 60 - DNA, 42 bp, Artificial sequence
  misc_feature (1)..(42): gene fragment
tcgactccagcacactacaccaccgcgtcgtcgagggtggtg